(19) United States
(12) Patent Application Publication
(10) Pub. No.: US 2023/0271176 A1
(43) Pub. Date: Aug. 31, 2023

(54) SPECIMEN COLLECTION TUBE SYSTEM AND METHOD

(71) Applicant: William J. Zinnanti, Santa Cruz, CA (US)

(72) Inventor: William J. Zinnanti, Santa Cruz, CA (US)

(21) Appl. No.: 18/195,083

(22) Filed: May 9, 2023

Related U.S. Application Data
(63) Continuation of application No. 16/877,013, filed on May 18, 2020, now abandoned.

(51) Int. Cl. B01L 3/00; A61B 10/02

(52) U.S. Cl. CPC B01L 3/5021; A61B 10/0233; A61B 2010/0216; B01L 2300/042; B01L 2300/047; B01L 2300/0832; B01L 2200/16

(57) ABSTRACT

A specimen collection tube for collection of biological or hazardous specimens with or without transfer media, collection means (swab, capillary tube, brush, or biopsy needle) and screw top cap. The proximal end of the collection means engages the proximal side of the specimen tube to secure the collection means at a given depth near the bottom of the tube, which may contain preservative, transfer, or wash media. The specimen collection tube has engagement means at the distal end to attach additional test tube for transfer and further processing of sample. The bottom of specimen collection tube is penetrable within the center of engagement means so that specimen can be transferred to test tube using centrifugation to force specimen through bottom as collection means is held in place.

### CROSS REFERENCE TO RELATED APPLICATIONS

This application is a continuation of U.S. patent application Ser. No. 16/877,013, filed May 18, 2020, which is hereby expressly incorporated herein by reference in its entirety for all purposes.

### TECHNICAL FIELD

The present invention relates to the field of laboratory equipment, specifically, specimen collection tubes and similar devices, particularly for use in biological and medical testing and assays, and related procedures.

### BACKGROUND OF THE INVENTION

Current specimen collection tubes are used as receptacles for collection of biological samples using swabs, capillary tubes, brushes, or biopsy needles (collection means). After the collection of biological samples using collection means, the distal end of the collection means in placed into the specimen tube usually containing preservative, transfer or wash media. The specimen tube is then transferred for further processing. Current available specimen tubes required the sample to be drawn back out from the proximal end of the tube, requiring additional handling with potential loss of sample and risk to operator to come in contact with sample especially if potentially infectious. This process will undoubtedly leave sample at the bottom of the tube within left over preservative, transfer or wash media. Therefore, transfer of entire sample is not achieved, and operator is at risk of coming into contact with potential hazardous of infectious sample due to additional handling. Additionally, when biological targets within biological samples are difficult to detect, more sample results in higher sensitivity and greater opportunity of detection.

Therefore, it is desirable, advantageous, cost saving and low risk to have a specimen collection tube with ability to transfer a biological or hazardous sample with minimal handling to second test tube for further processing. Here we describe a specimen collection tube with engagement means to secure collection means at the proximal end of specimen collection tube such that distal end of collection means is held at a given depth at the bottom of a specimen collection tube. This specimen collection tube has engagement means at distal end to attach second test tube for collection and further processing of the collected biological sample. The specimen collection tube has a frangible, penetrable bottom that remains intact with normal handling. The bottom of specimen collection tube is penetrable by sample using centrifugation to drive entire sample through specimen collection tube and into second test tube for collection of an entire sample with minimal handling. The secondary test tube can then be removed and closed until this test tube is used for further processing of sample.

### SUMMARY OF THE INVENTION

The present invention comprises, in part, a specimen collection tube assembly. A tube body is provided, having an open proximal end, and a closed end distal thereto. A cap is receivable on the open proximal end. A specimen retriever is receivable within the tube body when the cap is received on the open proximal end. The tube body includes a coupling structure disposed at the closed distal end which captures and holds a test tube brought into engagement therewith. The closed distal end of the tube body further includes a frangible section, which ruptures upon application of a force having a component directed along a direction from the open proximal end toward the closed distal end, such that material contained within the closed distal end of the tube is prompted to move into a test tube captured by the coupling structure.

In an embodiment of the invention, the cap is threadably couplable with the tube body.

In an embodiment of the invention, the coupling structure comprises a substantially cylindrical collar disposed about the frangible section, the cylindrical collar sized to be inserted into an end of a test tube. In an embodiment, the coupling structure engages a test tube with one of a friction fit, a threaded engagement, a snap-on engagement, a bayonet-type engagement.

In an embodiment, the frangible section comprises at least one line of weakness. In an embodiment, the at least one line of weakness extends at least one of radially from a bottommost reach of the tube body, extends in a circular path around a portion of the tube body.

In an embodiment, the specimen retriever comprises a stem having a proximal end and a distal end, with a grip disposed at the proximal end and a specimen-retaining tip disposed at the distal end. In an embodiment, the specimen-retaining tip comprises one of a swab, a capillary tube, a brush, a biopsy needle tip, and cannula.

In an embodiment, the grip is captured between an outer surface of the proximal end of the tube body, and an inner surface of the cap, when the cap is positioned on the proximal end of the tube body.

In an embodiment, the cap includes a region through which a specimen retriever may be thrust.

In an embodiment, a specimen collection solution is contained within the tube body and retained by the cap. In an embodiment, the specimen collection solution comprises at least one of a preservative, a transfer medium, a wash medium.

The present invention also comprises, in part, a specimen collection system comprising a specimen collection tube assembly comprising a tube body, having an open proximal end, and a closed end distal thereto; a cap receivable on the open proximal end; and a specimen retriever, receivable within the tube body, when the cap is received on the open proximal end. The tube body includes a coupling structure disposed at the closed distal end which captures and holds a test tube brought into engagement therewith. The closed distal end of the tube body further includes a frangible section, which ruptures upon application of a force having a component directed along a direction from the open proximal end toward the closed distal end, such that material contained within the closed distal end of the tube is prompted to move into a test tube captured by the coupling structure. A specimen collection solution is contained within the tube body and captured by the cap. A test tube is coupled to the closed distal end of the tube body.

The present invention also comprises in part a method of making a specimen collection system. The method comprises the steps of:
providing a specimen collection tube assembly, the specimen collection tube assembly comprising:
   a tube body, having an open proximal end, and a closed end distal thereto;
   a cap receivable on the open proximal end;
   a specimen retriever, receivable within the tube body, when the cap is received on the open proximal end;
   the tube body including a coupling structure disposed at the closed distal end which captures and holds a test tube brought into engagement therewith;
   the closed distal end of the tube body further Including a frangible section, which ruptures upon application of a force having a component directed along a direction from the open proximal end toward the closed distal end, such that material contained within the closed distal end of the tube body is prompted to move into a test tube captured by the coupling structure;
disposing a specimen collection solution within the tube body;
capturing the specimen collection solution within the tube body by attaching the cap to the open proximal end of the tube body; and
coupling a test tube to the closed distal end of the tube body.

The present invention also comprises, In part, a method for collecting a specimen comprising the steps of:
providing a specimen collection tube assembly, the specimen collection tube assembly comprising:
   a tube body, having an open proximal end, and a closed end distal thereto;
   a cap receivable on the open proximal end;
   a specimen retriever, receivable within the tube body, when the cap is received on the open proximal end;
   the tube body including a coupling structure disposed at the closed distal end which captures and holds a test tube brought into engagement therewith;
   the closed distal end of the tube body further including a frangible section, which ruptures upon application of a force having a component directed along a direction from the open proximal end toward the closed distal end, such that material contained within the closed distal end of the tube is prompted to move into a test tube captured by the coupling structure;
disposing a specimen collection solution within the tube body;
capturing the specimen collection solution within the tube body by attaching the cap to the open proximal end of the tube body; and
coupling a test tube to the closed distal end of the tube body;
grasping the specimen retriever and acquiring a specimen on a specimen-retaining tip on a distal end of the specimen retriever;
inserting the specimen retriever, distal end first, into to the tube body, such that the specimen retaining tip is immersed into the specimen collection solution;
placing the coupled tube body and test tube in a centrifuge;
actuating the centrifuge until rupture of the frangible section of the tube body occurs, causing movement of the specimen collection solution into the test tube;
removing the coupled tube body and test tube from the centrifuge.

An embodiment of the invention comprises a specimen collection tube assembly comprising:
a tube body, having an open proximal end, and a closed end distal thereto, a specimen collection solution contained within the closed distal end;
a cap receivable on the open proximal end;
a specimen retriever, receivable within the tube body, when the cap is received on the open proximal end;
the tube body including a coupling structure disposed at the closed distal end, the coupling structure insertable into an interior of an open end of a test tube brought into engagement therewith, to capture and hold the open end of the test tube;
the closed distal end of the tube body further including a frangible section, whereupon when the tube body is centrifuged, the frangible section will fail, and the specimen collection solution will exit the tube body through the distal end thereof.

In an embodiment of the invention, the cap and the tube body comprise mating thread structures.

In an embodiment of the invention, the frangible section comprises at least one line of weakness, the at least one line of weakness comprising at least one thinned region of a wall of the tube body in the distal end having a thickness that is less than a thickness of adjacent regions of the wall of the tube body. In such an embodiment, the at least one thinned region extends at least one of radially from a bottommost reach of the tube body, in a circular path around a portion of the tube body.

In an embodiment of the invention, the specimen retriever comprises a stem having a proximal end and a distal end, with a grip disposed at the proximal end and a specimen-retaining tip disposed at the distal end, wherein the specimen-retaining tip comprises one of a swab, a capillary tube, a brush, a biopsy needle tip, and cannula. The proximal end of the tube body may comprise a recess on an outer surface thereof, shaped to receive the grip of the specimen retriever, such that the grip can be captured between an outer surface of the proximal end of the tube body, and an inner surface of the cap, when the cap is positioned on the proximal end of the tube body.

In an embodiment of the specimen collection tube assembly, a test tube is coupled to the closed distal end of the tube body.

An embodiment of the invention comprises a method of making a specimen collection system comprising the steps of:
providing a specimen collection tube assembly, the specimen collection tube assembly comprising:
   a tube body, having an open proximal end, and a closed end distal thereto;
providing and containing a specimen collection solution within the distal end of the tube body;
   a cap receivable on the open proximal end;
   a specimen retriever, receivable within the tube body, when the cap is received on the open proximal end;
   the tube body including a coupling structure disposed at the closed distal end, the coupling structure insertable into an interior of an open end of a test tube brought into engagement therewith, to capture and hold the open end of the test tube;
   the closed distal end of the tube body further Including a frangible section, whereupon when the tube body is centrifuged, the frangible section will fail, and the specimen collection solution will exit the tube body through the distal end thereof;
capturing the specimen collection solution within the tube body by attaching the cap to the open proximal end of the tube body; and
coupling a test tube to the closed distal end of the tube body.

An embodiment of the invention comprises a method for collecting a specimen comprising the steps of:
providing a specimen collection tube assembly, the specimen collection tube assembly comprising:
   a tube body, having an open proximal end, and a closed end distal thereto;
providing and containing a specimen collection solution within the distal end of the tube body;
   a cap receivable on the open proximal end;
   a specimen retriever, receivable within the tube body, when the cap is received on the open proximal end;
   the tube body including a coupling structure disposed at the closed distal end, the coupling structure insertable into an interior of an open end of a test tube brought into engagement therewith, to capture and hold the open end of the test tube;
   the closed distal end of the tube body further including a frangible section, whereupon when the tube body is centrifuged, the frangible section will fail, and the specimen collection solution will exit the tube body through the distal end thereof;
capturing the specimen collection solution within the tube body by attaching the cap to the open proximal end of the tube body; and
coupling a test tube to the closed distal end of the tube body;
grasping the specimen retriever and acquiring a specimen on a specimen-retaining tip on a distal end of the specimen retriever;
inserting the specimen retriever, distal end first, into to the tube body, such that the specimen retaining tip is immersed into the specimen collection solution;
placing the coupled tube body and test tube in a centrifuge;
actuating the centrifuge until rupture of the frangible section of the tube body occurs, causing movement of the specimen collection solution into the test tube;
removing the coupled tube body and test tube from the centrifuge.

### BRIEF DESCRIPTION OF THE DRAWINGS

FIG. 1 illustrates a specimen collection tube, closed, with specimen retriever contained inside, according to an embodiment of the present invention.

FIG. 2 illustrates the specimen collection tube, including cap, and specimen retriever in position, according to the embodiment of FIG. 1.

FIG. 3 illustrates the specimen collection tube body, and specimen retriever, according to the embodiment of FIG. 1.

FIG. 4 is an enlarged view of an upper end of a specimen collection tube according to the embodiment of FIG. 1.

FIG. 5 illustrates a lower end of a specimen collection tube and a test tube for cooperation with same.

FIG. 6 illustrates the specimen collection tube and test tube of FIG. 5, wherein the test tube is coupled to the test tube receiving end of the specimen collection tube.

FIG. 7 illustrates the coupled specimen collection tube and test tube of FIG. 6, with the frangible end of the specimen collection tube breached following centrifugation.

FIG. 8 illustrates a swab tip for a specimen retriever according to an embodiment of the invention.

FIG. 9 illustrates a capillary tube tip for a specimen retriever according to an embodiment of the invention.

FIG. 10 illustrates a brush tip for a specimen retriever according to an embodiment of the invention.

FIG. 11 illustrates a biopsy needle tip for a specimen retriever according to an embodiment of the invention.

### DETAILED DESCRIPTION OF THE DRAWINGS

While this invention is susceptible of embodiment in many different forms, there are shown in the drawings and described in detail herein, specific embodiments, with the understanding that the present disclosure is to be considered as an exemplification of the principles of the invention, and is not intended to limit the invention to the embodiment(s) illustrated.

The invention and accompanying drawings will now be discussed in reference to the numerals provided therein to enable one skilled in the art to practice the present invention. The drawings and descriptions are exemplary of various aspects of the invention and are not intended to narrow the scope of the appended claims. Unless specifically noted, it is intended that the words and phrases in the specification and the claims be given their plain, ordinary and accustomed meaning to those of ordinary skill in the applicable arts. It is noted that the inventors can be their own lexicographers. The inventors expressly elect, as their own lexicographers, to use only the plain and ordinary meaning of terms in the specification and claims unless they dearly state otherwise and then further, expressly set forth the “special” definition of that term and explain how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the Inventors are fully informed of the standards and application of the special provisions of 35 U.S.C. § 112(f) or pre-AIA 35 U.S.C. § 112˜6. Thus, the use of the words “function,” “means” or “step” in the Detailed Description of the Invention or claims is not Intended to somehow indicate a desire to invoke the special provisions of 35 U.S.C. § 112(f) or pre-AIA 35 U.S.C. § 112˜6 to define the invention. To the contrary, if the provisions of 35 U.S.C. § 112(f) or pre-AIA 35 U.S.C. § 112˜6 are sought to be invoked to define the inventions, the claims will specifically and expressly state the exact phrases “means for” or “step for” and the specific function (e.g., “means for roasting”), without also reciting in such phrases any structure, material or act in support of the function, Thus, even when the claims recite a “means for . . . ” or “step for . . . ” if the claims also recite any structure, material or acts in support of that means or step, or that perform the recited function, then it is the clear intention of the inventor not to invoke the provisions of 35 U.S.C. § 112(f) or pre-AIA 35 U.S.C. § 112˜6. Moreover, even if the provisions of 35 U.S.C. § 112(f) or pre-AIA 35 U.S.C. § 112˜6 are invoked to define the claimed inventions, it is intended that the inventions not be limited only to the specific structure, material or acts that are described in the illustrated embodiments, but in addition, include any and all structures, materials or acts that perform the claimed function as described in alternative embodiments or forms of the invention, or that are well known present or later-developed, equivalent structures, material or acts for performing the claimed function.

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and apparatus are shown or discussed more generally in order to avoid obscuring the invention. In many cases, a description of the operation is sufficient to enable one to Implement the various forms of the invention, particularly when the operation is to be implemented in software. It should be noted that there are many different and alternative configurations, apparatus and technologies to which the disclosed inventions may be applied. Thus, the full scope of the inventions is not limited to the examples that are described below.

Various aspects of the present invention may be described in terms of functional block components and various processing steps. Such functional blocks may be realized by any number of hardware or software components configured to perform the specified functions and achieve the various results.

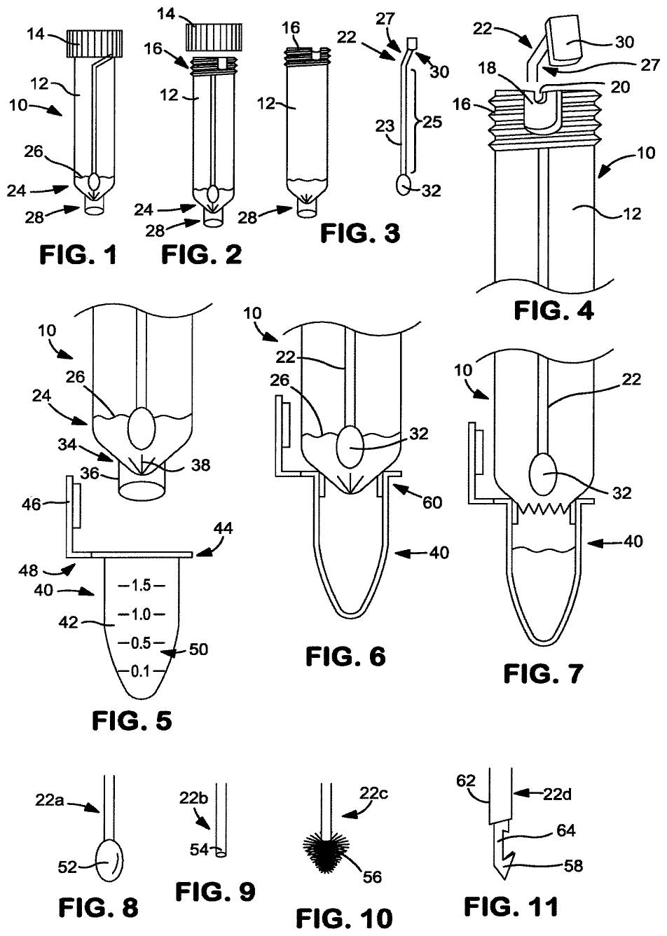

FIG. 1 illustrates a specimen collection tube assembly 10, closed, with a specimen retriever 22 contained inside, according to an embodiment of the present invention. Tube assembly 10 includes a body 12 and a cap 14. Tube body 12 may be fabricated from any suitable material, such as polypropylene, and may be fabricated by any suitable process, such as injection molding. Cap 14 may be fabricated from any suitable material, such as polypropylene, or may be fabricated from a harder material. Tube body 12 further includes a lower, sample preservation and transfer end 24, with a coupler 28 for connecting specimen collection tube assembly 10 to a test tube, such as test tube 40, described hereinafter with respect to FIGS. 5-7. Tube assembly 10 is shown in a configuration in which tube assembly 10 would be distributed in the field, having a quantity of a specimen collection solution 26 already sealed therewithin. Specimen collection solution 26 is at least one of a preservative, a transfer medium, a wash medium. By way of example, a preservative may be 70-100% ethanol or formalin; a transfer medium may be viral transfer media; a wash media may be 0.9% saline solution or ethanol; a lysis buffer may be made with phenol and guanidine isothiocyanate.

FIG. 2 Illustrates the specimen collection tube assembly 10, with cap 14 separated from body 12 to expose threads 16, including cap, and specimen retriever in position, according to the embodiment of FIG. 1. Specimen retriever 22 is shown in position within tube body 12. FIG. 3 illustrates specimen collection tube body 12, with specimen retriever 22 removed. Specimen retriever 22 includes a stem 23, a tab 30, and a tip 32. As will also be described with respect to FIG. 4, specimen retriever 22 includes stem 23, which has a lower straight portion 25 and an angled portion 27, terminating in tab or grip 30.

A lower end of specimen collection tube assembly 10 is illustrated in FIGS. 5-7. As previously described, tube body 12 Includes a lower, sample preservation and transfer end 24, with a coupler 28 for connecting specimen collection tube assembly 10 to a test tube, such as test tube 40. End 24, in an embodiment of the invention, includes a narrowing portion 34, which may be conical in shape. A connector collar 36 surrounds a frangible section 38. Frangible section 38 may be configured to include one or more lines of weakness, such as preformed lines where the material has been thinned, but not pierced or perforated, such that prior to collection of a specimen, the bottom of tube body 12 is substantially fluid (gas or liquid)-impermeable, and in any event is fabricated from material which will be impermeable to solution 26. Frangible section 38 is fabricated such that upon application of force in a longitudinal direction from top toward the bottom of tube body 12 against frangible section 38, it will be breached to permit fluid to leave tube body 12 via collar 36.

Collar 36 is shaped and sized to be able to receive, with a snug friction fit, the top end of a test tube 40, which may be an Eppendorf type test tube. Such a tube 40 is known in the art, and includes a body 42, an open top end 44, which may be surrounded by a radially outwardly projecting flange. A cap 46, which sealing engages top end 44, is joined to top end 44 by a web or living hinge 48. Tube 40 may further include volume indicia 50.

FIG. 4 is an enlarged view of an upper end of a specimen collection tube assembly 10, according to the embodiment of FIG. 1. As previously described a top end of tube body 12 includes a region of threads 16. Disposed within the region of threads 16 is a specimen retriever tab recess 18 and a specimen retriever positioning notch 20, collectively having dimensions such that upon insertion of specimen retriever 22 into tube body 12, tab 30 is fully received within recess 18, with the uppermost portion of stem angled portion 27 residing in notch 20. Because of the angled portion 27, the lower straight portion 25 of stem 23 is laterally spaced from an inner surface of tube body 12, and preferably substantially aligned with a central vertical longitudinal axis (not shown) of tube body 12.

FIGS. 5-7 illustrate sample preservation and transfer end 24 of specimen collection tube assembly 10, and test tube 40 for cooperation with same, and a method of using the specimen collection tube assembly 10 according to an embodiment of the invention. Referring to FIG. 5, to prepare assembly 10, a quantity of solution 26 is placed in tube body 12, together with a specimen retriever 22, and then cap 14 is tightly affixed. In a preferred embodiment, when cap 14 is in position, a seal is created, which, provided the cap is undisturbed, will persist for an extended period of time, under conditions defined by predetermined temperature, pressure and humidity ranges. Such lab supplies are expected to be kept in clean and dry conditions, at nominal “room temperature” for up to 12 months; however, this can be limited or impacted by specialized requirements for certain collection/transport media, which may sometimes require refrigeration.

In an embodiment of the invention, a test tube 40 is then coupled to collar 38 of tube assembly 10, to create a specimen collection kit 60, as shown in FIG. 6. As previously described, the connection between collar 36 and the interior of the opening at the top of test tube 40 may be a simple friction fit. At a location where specimens are being acquired, a technician will remove any external seal (not shown in the drawings) from the top of tube assembly 10, remove cap 14, extract specimen retriever 22, and collect the specimen, using whatever procedure is appropriate for the type of specimen being collected. The technician will then replace specimen retriever 22 within tube body 12, and close the cap, such that the specimen retriever tip 32 is again submerged in fluid 26, in its original position as shown in FIG. 6.

Upon collection of the specimen, at the time of conducting a test procedure, tube assembly 10 with coupled test tube 40 will be placed into a centrifuge (not shown). In an embodiment of the invention, the coupled tube assembly 10 and test tube 40 will be supported at one or more locations, e.g., at the bottom of test tube 40, at the radially projecting flange adjacent top end 44, and/or below threads 16 on tube body 12. As the centrifuge accelerates to operating speed, centripetal forces acting on fluid 26 will exert pressure on frangible section 38 and cause it to break, along the lines of weakness, thus opening the end of tube body 12, and allowing the fluid 26, with specimen materials, to move into test tube 40, as shown in FIG. 7.

In an embodiment of the invention, specimen retriever tip 32 will be a swab-type tip 52, coupled to a stem of a specimen retriever 22a, as shown in FIG. 8. In an alternative embodiment, a specimen retriever 22b is provided with a hollow stem defining a capillary tube 54, as shown in FIG. 9. FIG. 10 illustrates a specimen retriever 22c having a brush up 56. FIG. 11 illustrates a specimen retriever 22d having a tip in the form of a biopsy needle tip 58, having an outer cannula 62, which is slidable relative to an inner needle 64, which is provided with a specimen-entrapping recess 66, for capturing a specimen, in a manner similar to biopsy needles known in the art. These are examples of tips for a specimen retriever according to the invention; other tip shapes may be provided and employed by one having skill in the art, without departing from the scope of the invention.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

For example, instead of screw cap 14, test tube assembly 10 may be provided with a snap-on type cap (not shown). Further, whether the cap is a screw-on cap 14, or a snap fit, a breachable iris-type web or membrane may be provided on the cap, through which a specimen retriever as described herein, or a conventional long-stemmed specimen swab may be thrust, to position the swab tip into the fluid 26. With respect to the coupling between the specimen tube body and the test tube, as an alternative to the press- or friction-fit that has been shown or described, an arrangement of mating threads may be formed on the collar and the inner surface of the mouth of the test tube. As a still further alternative, a series of mating projections may be positioned on the outside of the collar and on the inside of the test tube, to create an interference-type snap-on fit. As a still further alternative, a bayonet-type arrangement may be provided. With regard to the frangible section 38, as illustrated, a plurality of lines of weakness may be provided in the closed end of tube body 12, wherein the lines extend radially from a bottommost reach of the tube body 12. In an alternative embodiment, the lines of weakness may be in the form of a single circle or a series of concentric circles. In another embodiment of the invention, the lines of weakness may have other regular or nonregular geometric shapes.

While the term “specimen tube” is used herein to describe the several embodiments of the invention, it is to be understood that the principles of the present invention may be applied to a wide variety of laboratory type containers having a range of shapes and configurations. Accordingly, the term “specimen tube” is to be construed in the broadest possible context as simply referring to a container for use in a laboratory or other setting for assaying, sampling, or other testing procedures.

Although the invention has been described with reference to the above examples, it will be understood that many modifications and variations are contemplated within the true spirit and scope of the embodiments of the invention as disclosed herein. Many modifications and other embodiments of the invention set forth herein will come to mind to one skilled in the art to which the invention pertains having the benefit of the teachings presented in the foregoing descriptions and the associated drawings. Therefore, it is to be understood that the invention shall not be limited to the specific embodiments disclosed and that modifications and other embodiments are intended and contemplated to be included within the scope of the appended claims. Although specific terms are employed herein, they are used in a generic and descriptive sense only and not for purposes of limitation.

What is claimed is:

1. A specimen collection tube assembly comprising:
a tube body, having an open proximal end, and a closed end distal thereto, a specimen collection solution contained within the closed distal end;
a cap receivable on the open proximal end;
a specimen retriever, receivable within the tube body, when the cap is received on the open proximal end;
the tube body including a coupling structure disposed at the closed distal end, the coupling structure insertable into an interior of an open end of a test tube brought into engagement therewith, to capture and hold the open end of the test tube;
the closed distal end of the tube body further including a frangible section, whereupon when the tube body is centrifuged, the frangible section will fail, and the specimen collection solution will exit the tube body through the distal end thereof.

2. The specimen collection tube assembly according to claim 1, wherein the cap is threadably couplable with the tube body.

3. The specimen collection tube assembly according to claim 1, wherein the coupling structure comprises a substantially cylindrical collar disposed about the frangible section.

4. The specimen collection tube assembly according to claim 3, wherein the coupling structure is configured to be inserted into and engage an interior surface of a test tube with one of a friction fit, a threaded engagement, a snap-on engagement, a bayonet type engagement.

5. The specimen collection tube assembly according to claim 1, wherein the frangible section comprises at least one line of weakness, the at least one line of weakness comprising a region of a wall of the tube body in the distal end having a thickness that is less than a thickness of adjacent regions of the wall of the tube body.

6. The specimen collection tube assembly according to claim 5, wherein the at least one line of weakness extends at least one of radially from a bottommost reach of the tube body, extends in a circular path around a portion of the tube body.

7. The specimen collection tube assembly according to claim 1, wherein the specimen retriever comprises a stem having a proximal end and a distal end, with a grip disposed at the proximal end and a specimen-retaining tip disposed at the distal end.

8. The specimen collection tube assembly according to claim 7, wherein the specimen-retaining tip comprises one of a swab, a capillary tube, a brush, a biopsy needle tip, and cannula.

9. The specimen collection tube assembly according to claim 7, wherein the proximal end of the tube body comprises a recess on an outer surface thereof, shaped to receive the grip of the specimen retriever, such that the grip can be captured between an outer surface of the proximal end of the tube body, and an inner surface of the cap, when the cap is positioned on the proximal end of the tube body.

10. The specimen collection tube assembly according to claim 1, wherein the cap includes a region through which a specimen retriever may be thrust.

11. The specimen collection tube assembly according to claim 1, wherein the specimen collection solution comprises at least one of a preservative, a transfer medium, a wash medium.

12. The specimen collection tube assembly according to claim 1, further comprising:
a test tube coupled to the closed distal end of the tube body.

13. The specimen collection system according to claim 12, wherein the test tube is coupled to the tube body with one of a friction fit, a threaded engagement, a snap-on engagement, a bayonet-type engagement.

14. The specimen collection system according to claim 12, wherein the specimen collection solution comprises at least one of a preservative, a transfer medium, a wash medium.

15. A method of making a specimen collection system comprising the steps of:
providing a specimen collection tube assembly, the specimen collection tube assembly comprising:
a tube body, having an open proximal end, and a closed end distal thereto;
providing and containing a specimen collection solution within the distal end of the tube body;
a cap receivable on the open proximal end;
a specimen retriever, receivable within the tube body, when the cap is received on the open proximal end;
the tube body including a coupling structure disposed at the closed distal end, the coupling structure insertable into an interior of an open end of a test tube brought into engagement therewith, to capture and hold the open end of the test tube;
the closed distal end of the tube body further including a frangible section, whereupon when the tube body is centrifuged, the frangible section will fail, and the specimen collection solution will exit the tube body through the distal end thereof;
capturing the specimen collection solution within the tube body by attaching the cap to the open proximal end of the tube body; and
coupling a test tube to the closed distal end of the tube body,

16. The method according to claim 15, wherein the step of coupling a test tube to the closed distal end of the tube body comprises coupling the test tube with one of a friction fit, a threaded engagement, a snap-on engagement, a bayonet-type engagement.

17. The method according to claim 15, wherein the specimen collection solution comprises at least one of a preservative, a transfer medium, a wash medium.

18. A method for collecting a specimen comprising the steps of:
providing a specimen collection tube assembly, the specimen collection tube assembly comprising:
a tube body, having an open proximal end, and a closed end distal thereto;
providing and containing a specimen collection solution within the distal end of the tube body;
a cap receivable on the open proximal end;
a specimen retriever, receivable within the tube body, when the cap is received on the open proximal end;
the tube body including a coupling structure disposed at the closed distal end, the coupling structure insertable into an interior of an open end of a test tube brought into engagement therewith, to capture and hold the open end of the test tube;
the closed distal end of the tube body further including a frangible section, whereupon when the tube body is centrifuged, the frangible section will fail, and the specimen collection solution will exit the tube body through the distal end thereof;
capturing the specimen collection solution within the tube body by attaching the cap to the open proximal end of the tube body; and
coupling a test tube to the closed distal end of the tube body;
grasping the specimen retriever and acquiring a specimen on a specimen-retaining tip on a distal end of the specimen retriever;
inserting the specimen retriever, distal end first, into to the tube body, such that the specimen retaining tip is immersed into the specimen collection solution;
placing the coupled tube body and test tube in a centrifuge;
actuating the centrifuge until rupture of the frangible section of the tube body occurs, causing movement of the specimen collection solution into the test tube;
removing the coupled tube body and test tube from the centrifuge.
